(19) United States
(12) Patent Application Publication
(10) Pub. No.: US 2024/0173273 A1
(43) Pub. Date: May 30, 2024

(54) USE OF PANTETHINE FOR THE TREATMENT OF SARS COV-2 INFECTIONS

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ PARIS CITE, Paris (FR); LEBANESE UNIVERSITY, Beirut (LB)

(72) Inventors: Mireille LAFORGE, Paris (FR); Mohamad ABOU HAMDAN, Beirut (LB); Lucien DE REGGI, Marseille (FR); Rachelle SALEH, Paris (FR)

(21) Appl. No.: 18/282,677

(22) PCT Filed: Mar. 23, 2022
(86) PCT No.: PCT/EP2022/057693

§ 371 Date: Sep. 18, 2023

(30) Foreign Application Priority Data
Mar. 24, 2021 (EP) 21305362.2

(51) Int. Cl. A61K 31/16; A61P 31/14

(52) U.S. Cl. CPC A61K 31/16; A61P 31/14

(57) ABSTRACT

SARS-COV and SARS-COV-2 share similar pathogenic pathways that interact with pathways of cellular cholesterol metabolism. Depletion of cholesterol from cell membranes reduced the infectivity of SARS-COV by 90%. Several studies reported that pantethine was able to reduce total cholesterol levels and total fatty acids synthesis but its anti-SARS COV-2 activity has never been investigated. The inventors now demonstrate that pantethine is highly effective in the control of SARS-COV-2 infections in vitro. In particular, the inventors show that the effect of pathethine on the inhibition of the replication of SARS-COV-2 is interested at the dose of 100 μM (inhibition is around 76%) and the most effective concentration is 1000 μM (inhibition is around 98%). Pantethine treatments were able to reduce significantly the expression of the viral Spike and Nucleocapsid proteins and the accessory proteins ORF6. Thus the present invention relates to the use of pantethine for the treatment of a SARS COV-2 infection.

### FIELD OF THE INVENTION

The present invention is in the field of medicine, in particular virology.

### BACKGROUND OF THE INVENTION

The newly identified Severe Acute Respiratory Syndrome Coronavirus 2 (SARS-COV-2) was declared as a pandemic by the World Health Organization (WHO) on Mar. 11, 2020. By March 2022, over 458 million people were infected worldwide with over 6 million reported deaths associated with COVID-19 [1]. This virus has become one of the major public health challenges in the world, with reports of new emerging variants that could escape a weak immune system and might be more contagious and deadlier [2; 3]. Despite the intense research effort, SARS-COV-2 still lacks a globally accepted antiviral drug which is urgent for effective and specific antiviral treatment.

One of the strategies to treat COVID-19 is to prevent the viral entry into permissive cells. The early steps of virus infection are initiated by the binding of the spike (S) protein to the functional entry receptor, the lipid raft protein angiotensin-converting enzyme 2 (ACE2) [4; 5; 6]. The disruption of lipid rafts prevents the correct exposure of ACE2, making it impossible for the viral S to dock [5; 6]. In addition, the subsequent stages of SARS-COV-2 lifecycle, e.g. activation, internalization and cell-to-cell transmission, rely on intact host lipid rafts to proceed. [7; 8] Thus, targeting host lipid rafts may be an effective strategy to reduce the infectivity of SARS-COV-2. This was experimentally shown in vitro for SARS-COV [6; 9], the close relative of SARS-COV-2.

SARS-COV and SARS-COV-2 share similar pathogenic pathways that interact with pathways of cellular cholesterol metabolism [10]. Depletion of cholesterol from cell membranes reduced the infectivity of SARS-COV by 90% [6]. A recent study reported that SARS-COV-2 needs cholesterol to invade host cells and form mega cells, but this need of cholesterol for infection was depending on membrane cholesterol of the virus but not the one of the host cell [11]. On the other hand, several studies have indicated that the capacity of enveloped viruses to infect host cells depends on a precise thiol/disulfide balance in their surface glycoprotein complexes, and any perturbations in this redox state affects virus/cell interactions [12; 12; 14; 15]. A recent study used molecular dynamics simulations to investigate the role of thiol-disulfide balance on the interactions between SARS-COV-2 spike proteins and ACE2. They observed a significant impairment of the binding affinity when all disulfide bonds of both ACE2 and SARS-CoV/CoV-2 spike proteins were reduced to thiol groups. Indeed, a contemporary work found that thiol-based drugs decreased the binding of SARS-COV-2 spike protein to its receptor, decreased the entry efficiency of SARS-COV-2 spike pseudotyped virus, and inhibited SARS-CoV-2 live virus infection. [16].

Pantethine, a naturally occurring low-molecular-weight thiol widely distributed in the living world, is the major precursor of coenzyme A, a cofactor in over 70 enzymatic pathways in the body. It has been used as a medicine in Japan for decades and is available in different countries as a nutritional supplement to help maintain balanced cholesterol levels and for healthy cardiovascular conditions. Pantethine exerts a broad range of protective activity in animal experiments and in clinical trials on humans, with few or no side effects. Several studies reported that pantethine was able to reduce total cholesterol levels and total fatty acids synthesis [17]. Pantethine was also able to alter lipid composition and cholesterol content of cell membrane rafts [18]. In addition, pantethine has been able to reduce microvesicle release in breast cancer cells [19], and in endothelial cells infected by Plasmodium berghei ANKA where this action was linked to the disulfide bonds in pantethine and its ability to influence various thiol-dependent mechanisms [20]. However, the interest of pantethine for inhibiting the replication of SARS COV-2 has never been investigated.

### SUMMARY OF THE INVENTION

The present invention is defined by the claims. In particular, the present invention relates to the use of pantethine for the treatment of a SARS COV-2 infection.

### DETAILED DESCRIPTION OF THE INVENTION

SARS-COV-2 interacts with cellular cholesterol during many stages of its life cycle (entry, internalization, budding and cell to cell transmission). Recently, cholesterol depletion from cell membranes was reported to significantly reduce SARS-COV-2 infection. In addition, thiol-based drugs were proposed to decrease SARS-COV-2 binding to its receptor in permissive cells. The low-molecular-weight thiol pantethine, the biologically active form of pantothenic acid (vitamin B5) and precursor of coenzyme A (CoA) which is an essential factor in lipid metabolism, was reported to reduce total cholesterol levels and fatty acids synthesis, and to potentially alter different processes that might be involve in SARS-COV-2 life cycle. Here, the inventor aim to investigate the possible anti-viral effects of pantethine in models of SARS-CoV-2 infection, which has not been studied yet.

Using different time-of-addition for pantethine treatments, they explored the potential anti-viral effects of pantethine in two in vitro experimental models of SARS-COV-2 infection, Vero E6 and Calu-3a cells. Infection was quantified by flow cytometry for the detection of infected cells positive for the viral spike protein, RT-qPCR for the detection of viral genes expression intracellularly and in the supernatant, and Western blot analysis to study the expression of spike and nucleocapsid proteins. The expression of ACE2, TMPRSS2 and different HECT E3 ligases expressions were also explored. Inflammation and sensing genes were also analysed in infected Calu-3a cells treated or not with pantethine.

Pantethine reduced significantly and dose dependently the infection of cells by SARS-COV-2 in both a pre-infection and post-infection treatment regimens. Accordingly, the intracellular expression of the viral Spike, Nucleocapsid and NSP6 proteins were highly reduced, and they observed a significant reduction of viral copy numbers in the supernatant of cells treated with pantethine. In addition, pantethine inhibited the infection-induced increase of TMPRSS2 and HECT E3 ligases expression in Vero E6 and Calu-3a cells as well as the immune-sensing and inflammatory genes in Calu-3a cells.

The first object of the present invention relates to a method of treating a SARS COV-2 infection in a subject in need thereof comprising administering to the subject a therapeutically effective amount of pantethine.

As used herein, the term “Severe Acute Respiratory Syndrome coronavirus 2” or “SARS-Cov-2” has its general meaning in the art and refers to the strain of coronavirus that causes coronavirus disease 2019 (“COVID-19”), a respiratory syndrome that manifests a clinical pathology resembling mild upper respiratory tract disease (common cold-like symptoms) and occasionally severe lower respiratory tract illness and extra-pulmonary manifestations leading to multi-organ failure and death. In particular, the term refers to the severe acute respiratory syndrome coronavirus 2 isolate 2019-nCOV_HKU-SZ-005b_2020 for which the complete genome is accessible under the NCBI access number MN975262.

In some embodiments, the subject can be human or any other animal (e.g., birds and mammals) susceptible to coronavirus infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the present invention relates to the treatment of a severe or critical form of SARS COV-2 infection.

As used herein, the term “severe or critical form of SARS COV-2 infection” refers to the progression of the disease to acute respiratory distress syndrome (ARDS), accountable for high mortality related to the damages of the alveolar lumen. Numerous patients with ARDS secondary to COVID-19 develop life-threatening thrombotic complications (5). More precisely severe form of COVID-19 can lead to critical illness, with acute respiratory distress (ARDS) and multiorgan failure as its primary complications, eventually followed by intravascular coagulopathy.

In some embodiments, the present invention also relates to the treatment of a long form of SARS COV-2 infection.

As used herein, the term “long form of SARS COV-2 infection” refers to the persistence of some symptoms weeks after having the disease: severe fatigue, neurological disorders (cognitive, sensory, headache), cardio-thoracic disorders (pain and chest tightness, tachycardia, dyspnea, cough) and disturbances of smell (anosmia), taste (ageusia), digestive disorders and skin symptoms

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pantethine” has its general meaning in the art and refers to the dimeric form of pantetheine, which is produced from pantothenic acid (vitamin B5) by the addition of cysteamine. The IUPAC name is (2R,2′ R)—N,N′-(3,12-Dioxo-7,8-dithia-4,11-diazatetradecane-1,14-diyl)bis(2,4-dihydroxy-3,3-dimethylbutanamide).

In some embodiments, therefore, the compound the present invention (i.e. pantethine) is administered to the subject before said subject is exposed to the SARS COV-2, during exposure to said virus or after exposure to said virus. Administration after exposure to the virus can be carried out at any time but will preferably be carried out as quickly as possible after exposure, in particular within 48 hours of the subject being exposed to said virus. Furthermore, it is also possible to envisage a plurality of successive administrations of the compound of the present invention, so as to increase the beneficial effects of the treatment. In order to increase the chances of cure, or at least prolong the life expectancy of the subject, or the prophylactic effect, it is possible in particular to carry out one or more successive administrations of said compound before the subject is exposed to the virus and/or during exposure to the virus and/or after exposure to the virus, in particular within 48 hours of said subject being exposed to said virus.

The compound of the present invention can be used to prevent, reduce and/or inhibit viral replication in a subject infected the SARS COV-2. The prevention or inhibition of viral replication can be either partial or total.

As used herein, the term “viral replication” includes the totality of the steps of the replication cycle of the virus. Especially this term includes the main steps of replication of the retroviruses described in the present application, including entry of the virus into the cell, and viral maturation.

In some embodiment, the compound of the present invention is administered to the subject in combination with at least one other therapeutic agent, preferably in combination with at least one other antiviral agent, more preferably in combination with at least one other antiviral agent selected from the group consisting of remdesivir, lopinavir, ritonavir, hydroxycholoroquine, and chloroquine. In some embodiments, the compound of the present invention is administered to the subject in combination with an interferon polypeptide. In some embodiments, the compound of the present invention is administered in combination with a corticosteroid.

As used herein, the term “therapeutically effective amount” meant a sufficient amount of the compound to treat a SARS COV-2 infection at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination with the specific agonist employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the compound of the present invention may be combined with pharmaceutically acceptable excipients, and optionally sustained-release matrices, such as biodegradable polymers, to form therapeutic compositions. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. Solutions comprising the compound of the invention as free base or pharmacologically acceptable salts can be prepared in water suitably mixed with a surfactant, such as hydroxypropyl cellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The compound of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to compound formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Toxicity test for Pantethine on Vero E6 cells

Véro E6 cells non infected (NI) or infected with the virus at MOI 0.05 and incubated with different concentrations of Pantethine (250 μM, 500 μM, 1000 μM, 1500 μm and 2000 μm) were collected at day 72 h post-infection from each well, washed twice with PBS before viability fixable dye staining for 30 min at 4ºC. Cells were washed after and fixed with 2% Paraformaldehyde (FPA) and then analyzed on a Fortessa Flux Cytometre. 30 000 events were recorded for each conditions in triplicate. Analyses were done later using a FlowJo Software and the percentages of viability were calculated according to the analysis report from the results of the triplicate of each condition. Results represent mean+SD (n=4) independent experiments with 3 independent point for each conditions separately.

FIG. 2: Effect of Pantethine on infection and mortality induce by SARS-COV-2. Flow cytometry analysis for the detection of Sars-COV-2 Spike protein in infected cells. A-B) Véro E6 cells non infected (NI) or infected with the virus at MOI 0.05 and incubated with different concentrations of Remedesivir (3.7 μM, 12 μM) or Pantethine (10 μM, 20 μM, 50 μM, 100 μM, 250 μM, 500 μM and 1000 μM) were collected at day 72 h post-infection from each well, and stained to mortality and infection rate. Analyses were done using a FlowJo Software. The % of infection is represented in each plot of analysis. C) Results represent mean+SEM of the % of of infection by analyzing the expression of the Spike protein in infected cells, for each condition of Pantethine treated or not treated cells. Comparisons differences between means were explored using One-way ANOVA test followed by Dunnett's post-hoc test. *** p 0.001 compared to the untreated group (n 3). D) Results represent mean+SEM of the % of inhibition of the expression of the Spike protein staining as compared to the untreated control group (n=3). Comparisons differences between means were explored using One-way ANOVA test followed by Dunnett's post-hoc test. *** p 0.001 compared to the untreated group.

FIG. 3: The antiviral activities of Pantethine against SARS-COV-2 in vitro. Spike and Nucleocapsid protein expression in infected cells.

Vero E6 cells were pre-treated with Pantethine at the indicated concentrations for 1 h, before the infection with the virus at MOI=0.05 for 72 h. Afterwards, the cells were cultured with drug-containing medium until the end of the experiment. A well with non-infected cells was performed as a negative control of the infection. At 72 h post-infection, cells were lysed by RIPA buffer and western blot analysis was performed to detect the expression of the Spike protein (S), the full length and S1 domain, and the Nucleocapsid protein (N). GAPDH was used as loading control. Results represent mean±SD, from 4 independent experiments (n=4) with 3 independent point per condition.

FIG. 4: The antiviral activities of Pantethine against SARS-COV-2 in vitro compared with Remdesivir at 72 h post-infection at MOI 0.05 in infected cells.

Vero E6 cells were pre-treated with 500 μM of Pantethine for 1 h, before the infection with the virus at MOI=0.05 for 72 h. Afterwards, the cells were cultured with drug-containing medium until the end of the experiment. A well with non-infected cells was performed as a negative control of the infection. At 72 h post-infection, cells were lysed by RIPA buffer and western blot analysis was performed to detect the expression of the Spike protein (S), the full length and S1 domain, and the Nucleocapsid protein (N). GAPDH was used as loading control. Results represent mean±SD, from 2 independent experiments (n=2) with 3 independent point per condition.

FIG. 5: The antiviral activities of Pantethine against SARS-COV-2 in vitro. Virus yield in the infected cell supernatants was quantified by qRT-PCR.

Vero E6 cells were pre-treated with Pantethine at the indicated concentrations for 1 h, before the infection with the virus at MOI=0.05. Afterwards, the cells were cultured with drug-containing medium until the end of the experiment. At 72 h post-infection, supernatants were collected, and viral RNA was extracted. Real-time PCR analysis was performed on supernatant using probes against either the SARS-COV-2 N and NSP6 genes. Results represent mean+SEM (n=3-6). Comparisons differences between means were explored using One-way ANOVA test followed by Dunnett's post-hoc test. *** p 0.001 compared to the untreated group.

FIG. 6: Pantethine Full-time treatment reduced SARS-COV-2 infection in Vero E6 cell cultures.

Full-time treatment with pantethine or remdesivir reduced significantly and dose dependently the infection of Vero E6 cells by SARS-COV-2 (MOI 0.05). A) Data represents the % of infection observed with the cytometry-analysis experiments and are shown as mean+SEM of results obtained from 3 independent experiments with 3 independent points per condition. B) Virus yields in supernatants of infected cells were quantified by qRT-PCR for the viral N gene. Calculated Ct values were converted to fold-reduction of samples compared to the non-infected cells using the ΔΔCt method (fold changed in viral RNA=2\hat{ }−ΔΔCt). Results represent mean+SEM. In all experiments, results were obtained from 4 independent experiments with 3 independent points per condition. *** p<0.001 compared to the control group (infected-untreated cells) by One-way ANOVA test followed by Dunnett's post-hoc test.

FIG. 7: Pantethine Post-entry treatment reduced SARS-COV-2 infection in Vero E6 cell cultures.

Post-entry treatment with pantethine or remdesivir reduced significantly and dose dependently the infection of Vero E6 cells by SARS-COV-2 (MOI 0.05). A) Data represents the % of infection observed with the cytometry-analysis experiments and are shown as mean+SEM of results obtained from 3 independent experiments with 3 independent points per condition. B) Virus yields in the supernatant of infected cells were quantified by qRT-PCR for the viral N gene. Calculated Ct values were converted to fold-reduction of samples compared to the supernatants non-infected cells using the ΔΔCt method (fold changed in viral RNA=2\hat{ }−ΔΔCt). Results represent mean+SEM. In all experiments, results were obtained from 4 independent experiments with 3 independent points per condition. *** p<0.001 compared to the control group (infected-untreated cells) by One-way ANOVA test followed by Dunnett's post-hoc test.

FIG. 8. Pantethine Pre-entry treatment reduced SARS-COV-2 infection in Vero E6 cell cultures.

Pre-entry treatment for 24 hrs with pantethine reduced significantly and dose dependently the infection of Vero E6 cells by SARS-COV-2 (MOI 0.05). A) Data represents the % of infection observed with the cytometry-analysis experiments and are shown as mean+SEM of results obtained from 3 independent experiments with 3 independent points per condition. B) Virus yields in the supernatant of infected cells were quantified by qRT-PCR for the viral N gene. Calculated Ct values were converted to fold-reduction of samples compared to the supernatants of non-infected cells using the ΔΔCt method (fold changed in viral RNA=2\hat{ }−ΔΔCt). Results represent mean+SEM. In all experiments, results were obtained from 3 independent experiments with 3 independent points per condition. ** p<0.01, *** p<0.001 compared to the control group (infected-untreated cells) by One-way ANOVA test followed by Dunnett's post-hoc test.

FIG. 9. Pantethine treatment reduced SARS-COV-2 infection in Calu-3a cell cultures. Full-time treatment with pantethine reduced significantly and dose dependently the infection of Calu-3a cells by SARS-COV-2 (MOI 0.05). A) Data represents the % of infection observed with the cytometry-analysis experiments and are shown as mean+SEM of results obtained from 3 independent experiments with 3 independent points per condition. B) Virus yields in supernatants of infected cells were quantified by qRT-PCR for the viral N gene. Calculated Ct values were converted to fold-reduction of samples compared to the non-infected cells using the ΔΔCt method (fold changed in viral RNA=2\hat{ }−ΔΔCt). Results represent mean+SEM. In all experiments, results were obtained from 4 independent experiments. *** p<0.001 compared to the control group (infected-untreated cells) by One-way ANOVA test followed by Dunnett's post-hoc test.

### EXAMPLE

### Methods

### Cells, Virus and Drugs

African green monkey kidney Vero E6 cell line was obtained kindly from Dr Andreola Marie-Aline, University of Bordeaux, and Calu-3a cells was obtained kindly from Dr. Pierre Olivier Vidalain, CIRI Lyon UMR 1087.

Cells were maintained in Eagle's medium (Dulbecco's modified Eagle's medium; Gibco Invitrogen supplemented with 10% heat-inactivated FBS, 1% PS (Penicillin 10,000 U/ml; Streptomycin 10,000 μg/ml) (Gibco Invitrogen) at 37° C. in a humidified atmosphere of 5% CO2. For the Calu-3a, Tryple express was used (Gibco 12604013). The strain BetaCoV/France/IDF0372/2020 was supplied by the National Reference Centre for Respiratory Viruses hosted by Institut Pasteur (Paris, France) and headed by Pr. Sylvie van der Werf. The human sample from which strain BetaCoV/France/IDF0372/2020 was isolated has been provided by Dr. X. Lescure and Pr. Y. Yazdanpanah from the Bichat Hospital, Paris, France. Moreover, the strain BetaCoV/France/IDF0372/2020 was supplied through the European Virus Archive goes Global (Evag) platform, a project that has received funding from the European Union's Horizon 2020 research and innovation program under grant agreement No 653316. The virus titer used for all the experiments was 3.75E+6 PFU/mL. All the infection experiments were performed in a biosafety level-3 (BLS-3) laboratory at the CRC (Cordelier Research Center). Pantethine was purchased from Sigma-Aldrich and Clinisciences (Cat no. HY-B1028) and Remdesivir from COGER (Cat no. AG--CR1-3713-M005).

### Evaluation of Antiviral Activities of the Drugs, Toxicity and Infection Inhibition

To evaluate the toxicity of the Pantethine on Vero E6 Cells and Calu-3a Cells and the antiviral efficacy, we measure by flux cytometry the mortality and the % of infected cells. Vero-E6 Cells were cultured overnight in 24-well cell-culture petridish with a density of 75×10⁴ cells/well, and Calu-3a cells were cultured 4 days before the experiments with a density of 150×10⁴ cells/well. Time of addition experiment was done as detailed in the next paragraph. Drugs were added each day at same concentration to cell culture. At 72 h post infection, the cell supernatant was collected and frozen immediately at −80° C. for viral extraction and q-PCR amplification. The cells were collected and a part was used to flux cytometry analysis to measure the inhibition of the infection by an intra-cellular staining against Spike protein (SARS-COV-2 Spike Protein-Alexa 647, Cat no. 51-6490-82, eBioscience) using a Cytofix/cytoperm fixation permeabilization kit (Cat no. 554714, BD) according to the manufacturer's instructions. Toxicity was analysed by using Viobility 405/452 Fixable Dye (Cat no. 130-109-814, from Miltenyi Biotec) according to the manufacturer's instructions. Briefly, the cells were washed twice with PBS before viability fixable dye staining for 30 min at 4° C. Then, the cells were permeabilized by the Cytofix/cytoperm buffer for 20 min, and after two washes with the permawash buffer, the anti-spike-Alexa 647 was added to the cells for 30 min at 4° C. After the staining, the cells were fixed with 2% Paraformaldehyde (FPA) and then analyzed on a Fortessa Flux Cytometer. 30 000 events were recorded for each conditions in triplicate. Analyses were done later using a FlowJo Software. The other part of the cells was lysed in RIPA lysis buffer (Invitrogen, Cat no. 10230544) containing protease (Roche) and phosphatase inhibitors (Invitrogen) for further quantification and immunoblotting analysis, or in LBP buffer for RNA purification and RT-qPCR analysis. Each condition was done in triplicate (n=3) in the same experiment and repeated for 3 independent experiments.

### Time-of-Addition Experiment—Pantethine Treatment Regimen

Pantethine and remdesivir were used for the time-of-addition experiment. Vero E6 cells (75×10³ cells/well) were treated with pantethine or Remdesivir at different stages of virus infection.

For “Full-time” treatment, cells were pre-treated with the drugs for 1 h prior to virus infection, followed by incubation with the virus for 2 h in the presence of the drugs until the end of the experiment.

For “Pre-Entry” treatment, the drugs were added to the cells for 1 h or 24 h before virus infection and maintained during the 2-h viral attachment process. Then, the virus-drug mixture was replaced with a fresh culture medium without drugs till the end of the experiment.

For “Post-entry” experiment, the virus was added to the cells to allow infection for 2 h, and then virus-containing supernatant was replaced with a drug-containing medium until the end of the experiment.

For all the experimental groups, cells were infected with the virus at an MOI of 0.05, and at 72 h p.i., cell supernatant and cell lysates were collected for qRT-PCR and Western blot analysis, respectively. Cells were also analyzed by flux cytometry for mortality and viral replication by analyzing the intracellular expression of the spike protein. For Calu-3a cells, only a “Full-time” treatment was used in all the experiments.

Viral RNA Extraction and Quantitative Real-Time RT-PCR (qRT-PCR)
1. Viral RNA Extraction from the Supernatant

Two hundred microliter cell culture supernatant was harvested for viral RNA extraction using the MiniBEST Viral RNA/DNA Extraction Kit (Takara, Cat no. 9766) according to the manufacturer's instructions. RNA was eluted in 30 μL RNAase Free water.

### 2. Intracellular RNA Purification

After being washed with PBS, cells were lysed with LBP and stocked at −80° C. ARN purification was done using “Nucleospin RNA PLUS” kit according to the manufacturer's recommended procedures (Machery Nagel ref #740984.250)

3. Quantitative Real-Time RT-PCR (qRT-PCR)

Total RNA was converted to cDNA using PrimeScript RT Reagent Kit with gDNA Eraser (Takara, Cat no. RR047A), following the manufacturer's recommended procedures. Quantitative PCR was performed using TB Green Premix Ex Taq II (Takara Cat no. RR820A). Briefly, each reaction consisted of a total volume of 25 μl containing 1 μL of each primer [0.4 μM/μL], 2 μl of cDNA (5 ng/μL), 12.5 μl TB Green Premix Ex Taq II and 8.5 μL of Rnase free Water.

Real-time PCR was performed using Bio Rad CFX384 Real-Time system PCR Machine. The thermal cycling conditions used were as follows: initial denaturation: 95° C. for 30 s, followed by 40 cycles of amplification at 96° C. for 5 seconds, and 60° C. for 30 seconds. The primers used for SARS-COV-2 N, NSP6 genes designed and described by Abdel-Sater et al (1) were purchased from Eurofins.

| | | |
|---|---|---|
| N | Fw | CGTTTGGTGGACCCTCAGAT |
| | | (SEQ ID NO: 1) |
| | Rv | CCCCACTGCGTTCTCCATT |
| | | (SEQ ID NO: 2); |
| NSP6 | Fw | GGTTGATACTAGTTTGTCTGGTTTT |
| | | (SEQ ID NO: 3); |
| | Rv | AACGAGTGTCAAGACATTCATAAG |
| | | (SEQ ID NO: 4). |
| GAPDH | Fw | AAGGTCGGAGTCAACGGATTT |
| | | (SEQ ID NO: 5) |
| | Rv | TGAAGGGGTCATTGATGGCA |
| | | (SEQ ID NO: 6) |
| WWWP1 | Fw | TGTAAATGTTACGCCACAGACT |
| | | (SEQ ID NO: 7) |
| | Rv | GCTTGTTTCAAATCTATCGTTGC |
| | | (SEQ ID NO: 8) |
| WWWP2 | Fw | GAAAGTGGTGTCCGCAAAGC |
| | | (SEQ ID NO: 9) |
| | Rv | ATGACTCTGTGCCGTGACATT |
| | | (SEQ ID NO: 10) |
| NEDD4 | Fw | CTGCTACGGACAATTATACCCTA |
| | | (SEQ ID NO: 11) |
| | Rv | CATCCAACAGTTTGCCATGATA |
| | | (SEQ ID NO: 12) |
| NEDD4-L | Fw | ACGTAGCGGATGAGAATAGAGAAC |
| | | (SEQ ID NO: 13) |
| | Rv | CTGTGATTAGATGGGTTTACCCTGA |
| | | (SEQ ID NO: 14) |
| SMURF1 | Fw | CCGCTCCAAGGCTTCAAGG |
| | | (SEQ ID NO: 15) |
| | Rv | ATCCGGTTAAAGCAGGTATGGG |
| | | (SEQ ID NO: 16) |
| ACE-2 | Fw | GGGATCAGAGATCGGAAGAAGAAA |
| | | (SEQ ID NO: 17) |
| | Rv | AGGAGGTCTGAACATCATCAGTG |
| | | (SEQ ID NO: 18) |
| TMPRSS2 | Fw | AATCGGTGTGTTCGCCTCTAC |
| | | (SEQ ID NO: 19) |
| | Rv | CGTAGTTCTCGTTCCAGTCGT |
| | | (SEQ ID NO: 20) |
| STING | Fw | TACATCGGATATCTGCGGCTG |
| | | (SEQ ID NO: 21) |
| | Rv | CGGTCTGCTGGGGCAGTTTATC |
| | | (SEQ ID NO: 22) |
| IFN-β | Fw | GGCACAACAGGTAGTAGGCG |
| | | (SEQ ID NO: 23) |
| | Rv | AAGCCTCCCATTCAATTGCC |
| | | (SEQ ID NO: 24) |
| IL-6 | Fw | GAGAAAGGAGACATGTAACAAGAG |
| | | (SEQ ID NO: 25) |
| | Rv | CCTCTTTGCTGCTTTCACAC |
| | | (SEQ ID NO: 26) |
| MAVS | Fw | CCGAGTCTCGTTTCCTCTC |
| | | (SEQ ID NO: 27) |
| | Rv | CTGAAATTGCGGCAGATATAC |
| | | (SEQ ID NO: 28) |
| IRF3 | Fw | CTGATACCCAGGAAGACATTC |
| | | (SEQ ID NO: 29) |
| | Rv | GGGCCAACACCATGTTAC |
| | | (SEQ ID NO: 30) |

SARS-COV-2 cDNA (Ct˜20 for N and NSP6 genes) was used as a positive control. for viral gene expression in the supernatant. Calculated Ct values were converted to fold-reduction of treated samples compared to control using the ΔCt method (fold changed in viral RNA=2\hat{ }ΔCt). Each experiment was repeated 3 times.

### Western Blot Analysis

For Western blot analysis, 40 μg of proteins from each point were resolved on 4-12% NUPAGE SDS-PAGE (Invitrogen) and then transferred onto nitrocellulose membranes (Amersham Bioscience). After being blocked with 5% BSA in TBS buffer containing 0.05% Tween 20, the membranes were then probed with the mouse anti-Spike antibody (S1-NTD) (E7M5X) (1/2000, Ozyme, Cat. No. 42172S) and the anti-N antibody (1:10 000 dilution, Fisher scientific, Cat. No. MA536086) on primary antibodies and the horseradish peroxidase (HRP)-conjugated Goat-Anti-Mouse IgG or Goat-Anti-Rabbit IgG (Invitrogen) as the secondary antibody, respectively. Protein bands were detected by ECL Chemiluminescent substrate (Pierce) using a CCD camera (Syngene Pxi-4).

### Immunocytochemistry and Confocal Microscopy

At the end of incubation times, cells were fixed with 4% PFA for 20 min at RT. Cells were washed in PBS and incubated for 30 min in PBS with 5% normal donkey serum (NDS, Sigma-Aldrich) and 0.1% Triton X-100. Primary antibody (1:1000; SARS-COV/SARS-COV-2 (COVID-19) spike antibody [1A9] Gene Tex, CA 92606 USA) was diluted in PBS with 1% NDS and 0.1% Triton X-100 and incubated overnight at 4ºC. Cells were washed in PBS and subsequently incubated with a secondary antibody Alexa Fluor 488 (A488)-conjugated donkey anti-rabbit (1:300; Life Technologies, Molecular Probes) diluted in PBS with 1% NDS and 0.1% Triton X-100 for 1 hr at RT. Cells were rinsed in PBS, stained with DAPI 1:1000 in PBS for 2 mins at RT, rinsed, and incubated with PBS for confocal microscopic analysis. Cells were analyzed using a Leica TCS SP8 confocal scanning system (Leica Microsystems, Wetzlar, Germany) equipped with 405-nm Diode, 488-nm Ar, 561-nm DPSS, and 633-nm HeNe lasers. Eight-bit digital images were collected from a single optical plane using 40×HC PL APO CS2 oil-immersion Leica objective (numerical aperture 1.30). For each optical section, double-fluorescence images were acquired in sequential mode to avoid potential contamination by linkage-specific fluorescence emission cross-talk. Settings for laser intensity, beam expander, pinhole (1 Airy unit), range property of emission window, electronic zoom, gain and offset of photomultiplicator, field format, scanning speed were optimized initially and held constant throughout the study so that all sections were digitized under the same conditions. Composite illustrations were built-in Adobe Photoshop CS3 (Adobe Systems, San Jose, CA, USA).

### Statistics Analysis

Statistics analysis between means were explored using One-way ANOVA test followed by Dunnett's post-hoc test to determine significance was performed using GraphPad Prism software (GraphPad Software Inc., USA). Values are given as means±S.E.M. and a p-value <0.05% was considered significant.

### Example 1

### Results:

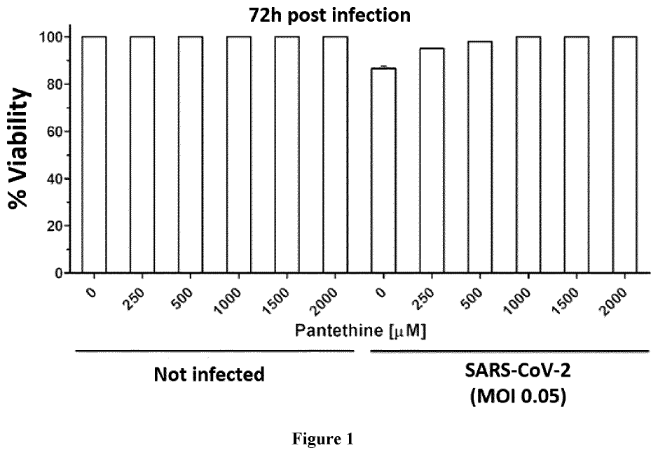

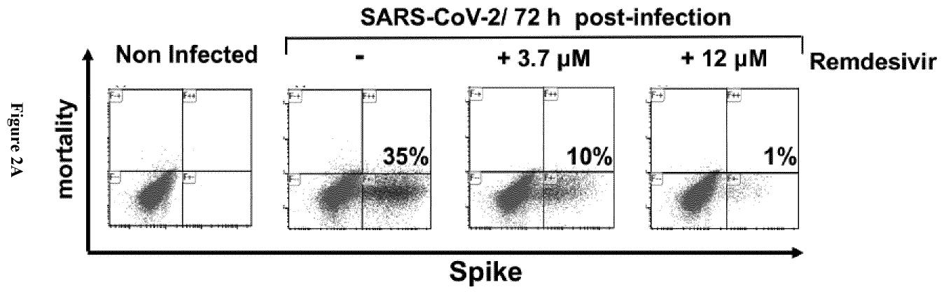

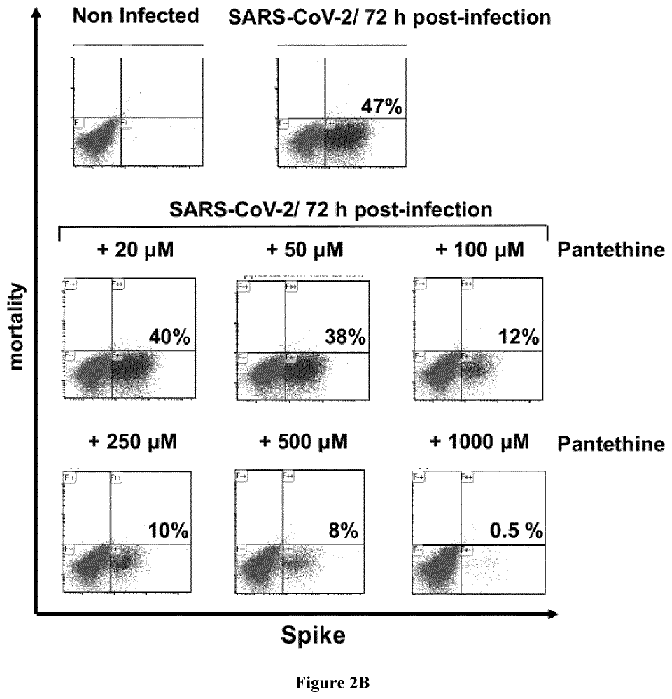

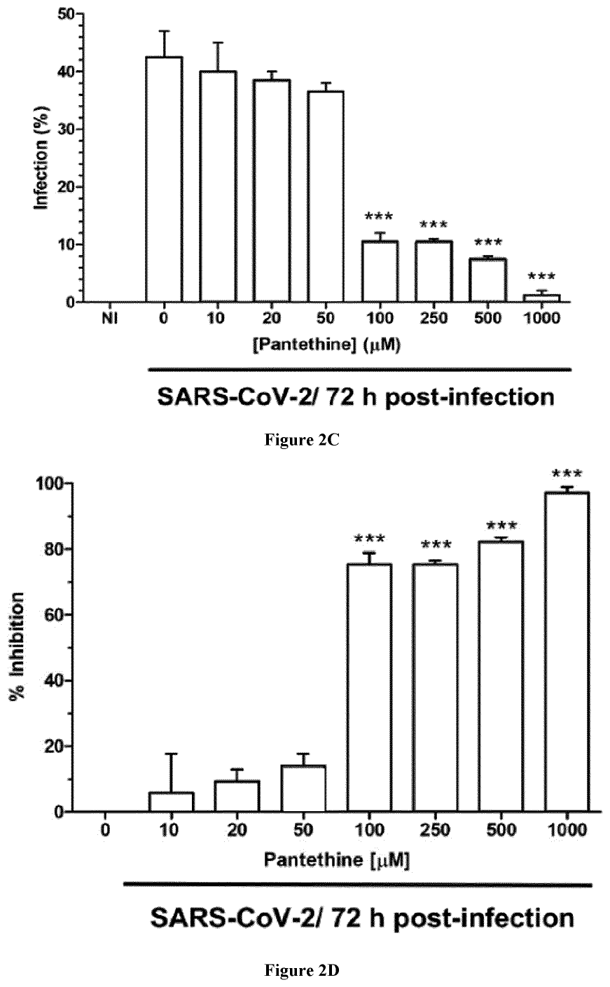

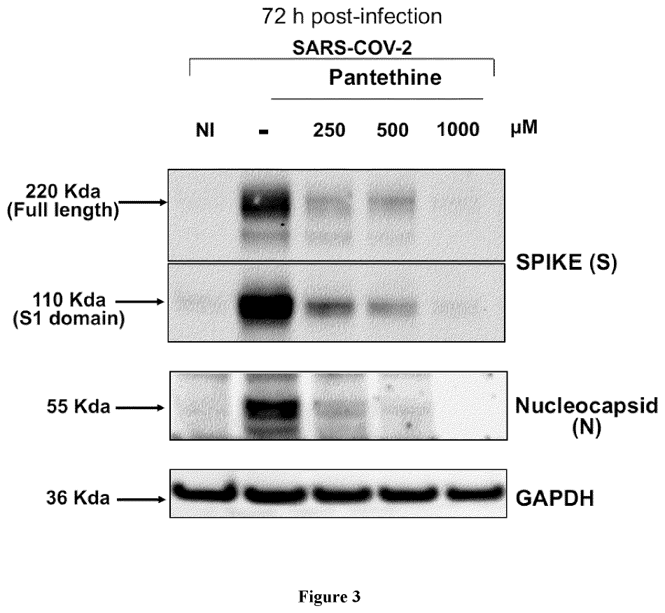

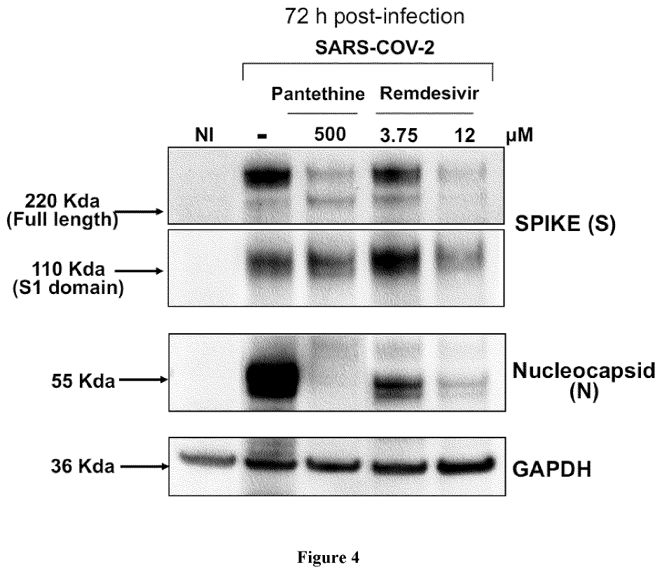

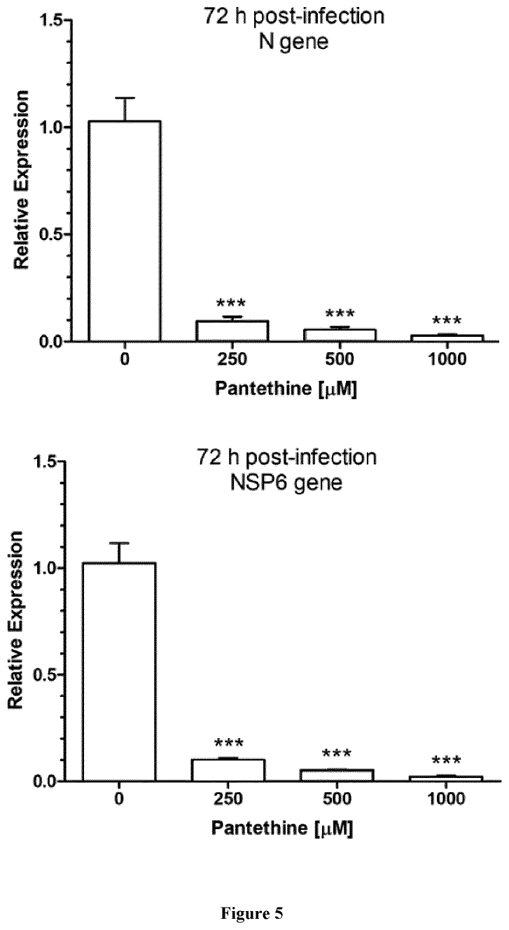

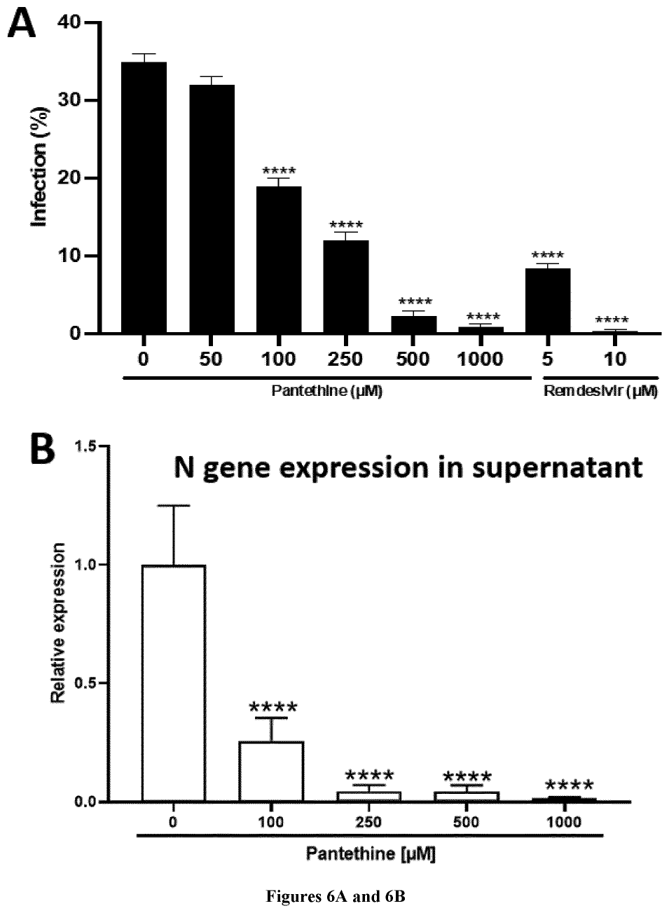

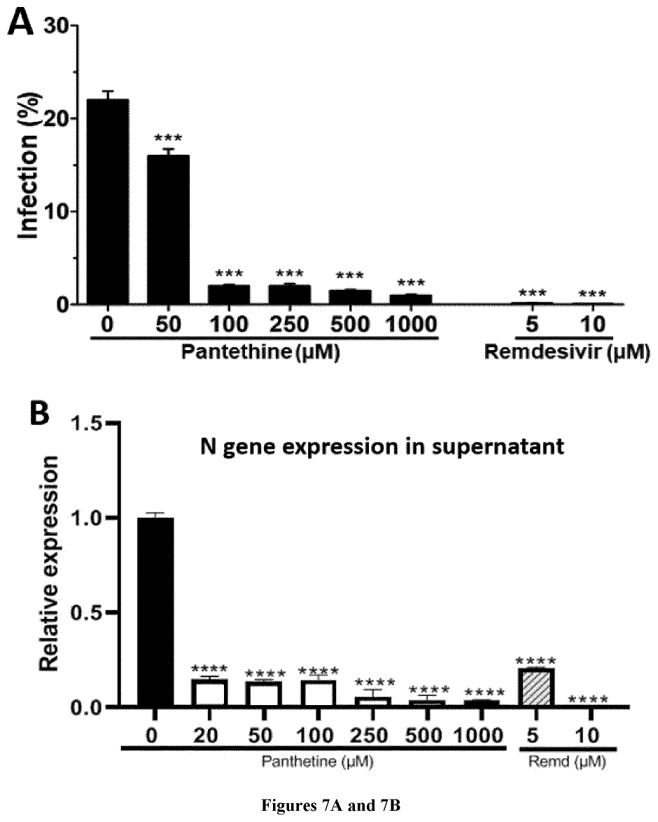

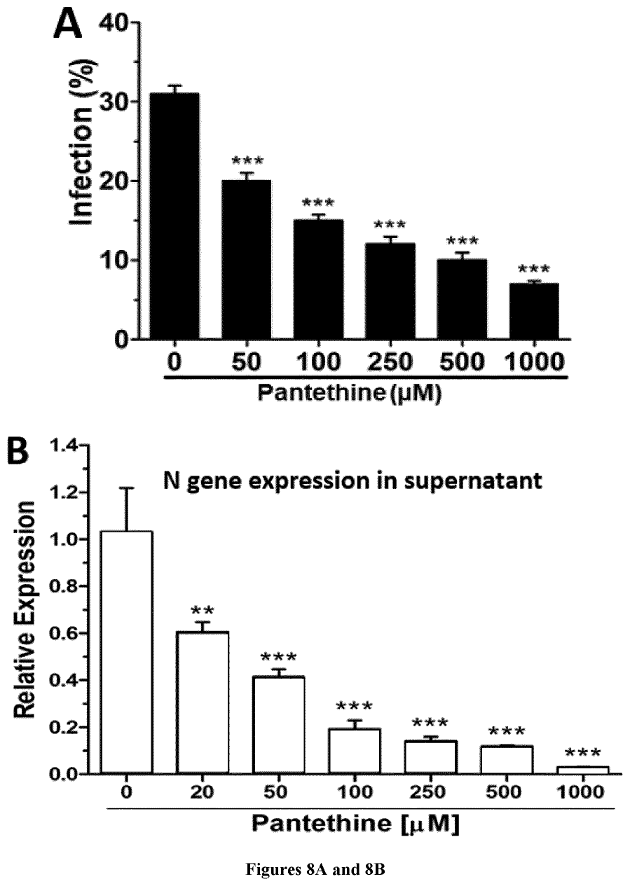

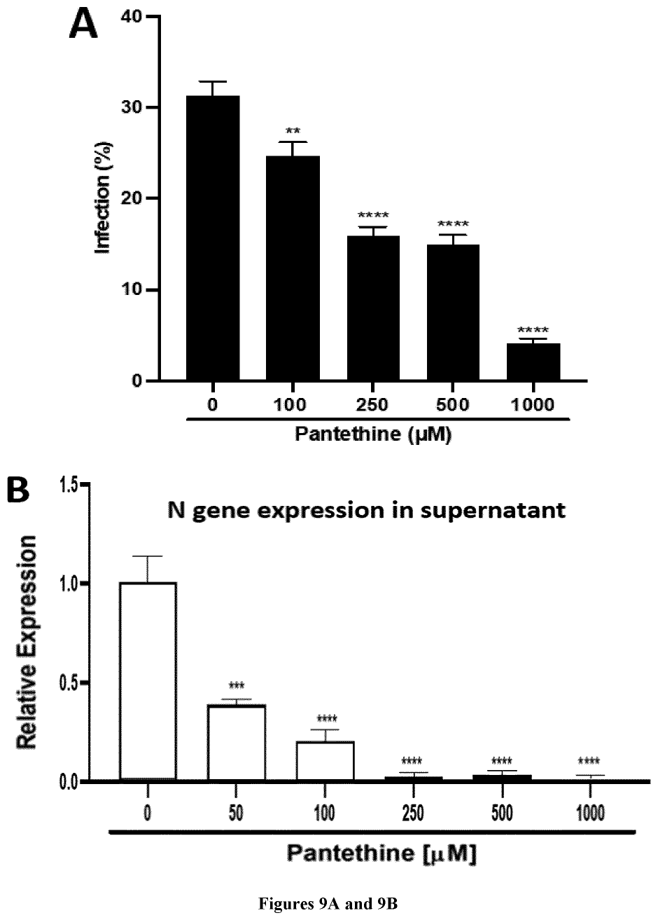

Treatments with different concentrations of pantethine had no significant toxic effect on Vero cells whether they were infected or not with the virus at MOI 0.05 for 72 h post-infection (FIG. 1).

The antiviral and mortality effects of different concentrations of pantethine were evaluated by flux cytometry analysis using intracellular staining against SARS-Cov-2 spike proteins and a dye of viability. Remdesivir was used as positive control of our analysis. Results show that the effect of pathethine on the inhibition of the replication of SARS-COV-2 is interested at the dose of 100 μM (inhibition is around 76%) and the most effective concentration is 1000 μM (inhibition is around 98%) (FIG. 2A,B,C,D).

The antiviral effects of different concentrations of pantethine were also evaluated by western blot, studying the expression of the viral Spike and Nucleocapsid proteins inside the infected cells, and by qRT-PCR measuring the virus yield in the supernatants of infected cells (expression of the viral N and NSP6 genes).

Pantethine treatments were able to reduce significantly the expression of the viral Spike and Nucleocapsid proteins within infected cells (FIG. 3), this reduction was comparable to that obtained with Remdisivir (FIG. 4). Indeed, Pantethine treatments were able to reduce significantly the relative expression of the structure protein the Nucleocapsid (N) and the accessory protein ORF6 (NSP6) genes in the supernatants of infected cells (FIG. 5).

Our findings demonstrate that pantethine is highly effective in the control of SARS-COV-2 infections in vitro.

### Example 2

### Results:

Full-time treatment with pantethine or remdesivir reduced significantly and dose dependently the infection of Vero E6 cells by SARS-COV-2 (MOI 0.05). Indeed, pantethine Full-time treatment reduced SARS-COV-2 infection in Vero E6 cell cultures (FIGS. 6A et 6B). Post-entry treatment with pantethine or remdesivir reduced significantly and dose dependently the infection of Vero E6 cells by SARS-COV-2 (MOI 0.05). Indeed, pantethine Post-entry treatment reduced SARS-COV-2 infection in Vero E6 cell cultures (FIGS. 7A et 7B). Pre-entry treatment for 24 hrs with pantethine reduced significantly and dose dependently the infection of Vero E6 cells by SARS-COV-2 (MOI 0.05). Pantethine Pre-entry treatment for 24 hrs reduced SARS-COV-2 infection in Vero E6 cell cultures (FIGS. 8A et 8B).

Full-time treatment with pantethine reduced significantly and dose dependently the infection of Calu-3a cells by SARS-COV-2 (MOI 0.05). Indeed, pantethine treatment reduced SARS-COV-2 infection in Calu-3a cell cultures (FIGS. 9A et 9B).

Results also show that pantethine decreased the HECT E3 ligase-increase induced by SARS-CoV-2 infection in vitro and reduced the inflammatory response induced by SARS-COV-2 in Calu-3a cells (data not shown). Moreover, pantethine effects on ACE2 and TMPRSS2 expression in SARS-COV-2 infected cells (data not shown).

Finally, the results show that pantethine treatment had no effects on HECT E3 liagses expression in non-infected Vero E6 and Calu-3a cells and pantethine treatment had no significant effect on ACE2 and TMPRSS2 expression in non-infected Vero E6 and Calu-3 cells (data not shown).

To conclude, the results demonstrate that pantethine was significantly effective in controlling SARS-COV-2 infection in two experimental models and might present a new antiviral drug by repositioning for the prevention or treatment of COVID-19.

### REFERENCES

Throughout this application, various references describe the state of the art to which this invention pertains. The disclosures of these references are hereby incorporated by reference into the present disclosure.
1. Worldometer.info/Coronavirus updates. https://www.worldometers.info/coronavirus/#countries
2. Andreano E—December 2020-bioRxiv-SARS-COV-2 escape in vitro from a highly neutralizing COVID-19 convalescent plasma-nihpp-2020.12.28.424451
3. Weisblum 2020—elife-Escape from neutralizing antibodies by SARS-COV-2 variants
4. Hoffmann M, Kleine-Weber H, Schroeder S, Krüger N, Herrler T, Erichsen S, et al. SARS-COV-2 cell entry depends on ACE2 and TMPRSS2 and is blocked by a clinically proven protease inhibitor. Cell. (2020) 181:271-80.e278. doi: 10.1016/j.cell.2020.02.052
5. Glende J, Schwegmann-Wessels C, Al-Falah M, Pfefferle S, Qu X, Deng H, et al. Importance of cholesterol-rich membrane microdomains in the interaction of the S protein of SARS-coronavirus with the cellular receptor angiotensin-converting enzyme 2. Virology. (2008) 381:215-21. doi: 10.1016/j.virol.2008.08.026.
6. Lu Y, Liu D X, Tam J P. Lipid rafts are involved in SARS-COV entry into Vero E6 cells. Biochem Biophys Res Commun. (2008) 369:344-9. doi: 10.1016/j.bbrc.2008.02.023
7. Wang H, Yang P, Liu K, Guo F, Zhang Y, Zhang G, et al. SARS coronavirus entry into host cells through a novel clathrin- and caveolae-independent endocytic pathway. Cell Res. (2008) 18:290-301. doi: 10.1038/cr.2008.15
8. Musarrat F, Chouljenko V, Dahal A, Nabi R, Chouljenko T, Jois S D, et al. The anti-HIV drug nelfinavir mesylate (Viracept) is a potent inhibitor of cell fusion caused by the SARSCOV-2 spike (S) glycoprotein warranting further evaluation as an antiviral against COVID-19 infections. J Med Virol. (2020). doi: 10.1002/jmv.25985. [Epub ahead of print].
9. Li G M, Li Y G, Yamate M, Li S M, Ikuta K. Lipid rafts play an important role in the early stage of severe acute respiratory syndrome-coronavirus life cycle. Microbes Infect. (2007) 9:96-102. doi: 10.1016/j.micinf.2006.10.015
10. Ballout R A, Sviridov D, Bukrinsky M I, Remaley A T. The lysosome: a potential juncture between SARS-COV-2 infectivity and Niemann-Pick disease type C, with therapeutic implications. FASEB J. (2020) 34:7253-64. doi: 10.1096/fj.202000654R.
11. David W. Sanders, Chanelle C. Jumper, Paul J. Ackerman, Dan Bracha, Anita Donlic, Hahn Kim, Devin Kenney, Ivan Castello-Serrano, Saori Suzuki, Tomokazu Tamura, Alexander H. Tavares, Mohsan Saeed, Alex S. Holehouse, Alexander Ploss, Ilya Levental, Florian Douam, Robert F. Padera, Bruce D. Levy, Clifford P. Brangwynne. SARS-COV-2 Requires Cholesterol for Viral Entry and Pathological Syncytia Formation. doi: https://doi.org/10.1101/2020.12.14.422737
12. Ryser, H. J.; Levy, E. M.; Mandel, R.; DiSciullo, G. J. Inhibition of human immunodeficiency virus infection by agents that interfere with thiol-disulfide interchange upon virus-receptor interaction. Proc. Natl. Acad. Sci. U.S.A. 1994, 91, 4559-4563.
13. Mathys, L.; Balzarini, J. The role of cellular oxidoreductases in viral entry and virus infection-associated oxidative stress: potential therapeutic applications. Expert Opin. Ther. Targets 2016, 20, 123-143.
14. Sanders, D. A. (2000). Sulfhydryl involvement in fusion mechanisms. Subcell Biochem 34, 483-514
15. Gallagher, T. M. (1996). Murine coronavirus membrane fusion is blocked by modification of thiols buried within the spike protein. J Virol 70, 4683-4690.
16. Sanchita Hati and Sudeep Bhattacharyya ACS Omega 2020, 5, 16292-16298
17. Evans M, Rumberger J A, Azumano I, Napolitano J J, Citrolo D, Kamiya T. Pantethine, a derivative of vitamin B5, favorably alters total, LDL and non-HDL cholesterol in low to moderate cardiovascular risk subjects eligible for statin therapy: a triple-blinded placebo and diet-controlled investigation. Vasc Health Risk Manag. 2014 Feb. 27; 10:89-100. doi: 10.2147/VHRM.S57116. PMID: 24600231; PMCID: PMC3942300.
18. van Gijsel-Bonnello M, Acar N, Molino Y, Bretillon L, Khrestchatisky M, de Reggi M, Gharib B. Pantethine Alters Lipid Composition and Cholesterol Content of Membrane Rafts, With Down-Regulation of CXCL12-Induced T Cell Migration. J Cell Physiol. 2015 October; 230(10):2415-25. doi: 10.1002/jcp.24971. PMID: 25728249.
19. Roseblade A, Luk F, Ung A, Bebawy M. Targeting microparticle biogenesis: a novel approach to the circumvention of cancer multidrug resistance. Curr Cancer Drug Targets. 2015; 15(3):205-14. doi: 10.2174/1568009615666150225121508. PMID: 25714701.
20. Penet M F, Abou-Hamdan M, Coltel N, Cornille E, Grau G E, de Reggi M, Gharib B. Protection against cerebral malaria by the low-molecular-weight thiol pantethine. Proc Natl Acad Sci USA. 2008 Jan. 29; 105(4):1321-6. doi: 10.1073/pnas.0706867105. Epub 2008 Jan. 14. PMID: 18195363; PMCID: PMC2234136.

1. A method of treating a SARS COV-2 infection in a subject in need thereof comprising administering to the subject a therapeutically effective amount of pantethine.

2. The method of claim 1 wherein pantethine is administered to the subject before said subject is exposed to the SARS COV-2, during exposure to said virus or after exposure to said virus.


SEQUENCE LISTING (30 sequences)

SEQ ID NO: 1 - DNA, 20 bp, Artificial
  Synthetic primer
cgtttggtggaccctcagat

SEQ ID NO: 2 - DNA, 19 bp, Artificial
  Synthetic primer
ccccactgcgttctccatt

SEQ ID NO: 3 - DNA, 25 bp, Artificial
  Synthetic primer
ggttgatactagtttgtctggtttt

SEQ ID NO: 4 - DNA, 24 bp, Artificial
  Synthetic primer
aacgagtgtcaagacattcataag

SEQ ID NO: 5 - DNA, 21 bp, Artificial
  Synthetic primer
aaggtcggagtcaacggattt

SEQ ID NO: 6 - DNA, 20 bp, Artificial
  Synthetic primer
tgaaggggtcattgatggca

SEQ ID NO: 7 - DNA, 22 bp, Artificial
  Synthetic primer
tgtaaatgttacgccacagact

SEQ ID NO: 8 - DNA, 23 bp, Artificial
  Synthetic primer
gcttgtttcaaatctatcgttgc

SEQ ID NO: 9 - DNA, 20 bp, Artificial
  Synthetic primer
gaaagtggtgtccgcaaagc

SEQ ID NO: 10 - DNA, 21 bp, Artificial
  Synthetic primer
atgactctgtgccgtgacatt

SEQ ID NO: 11 - DNA, 23 bp, Artificial
  Synthetic primer
ctgctacggacaattatacccta

SEQ ID NO: 12 - DNA, 22 bp, Artificial
  Synthetic primer
catccaacagtttgccatgata

SEQ ID NO: 13 - DNA, 24 bp, Artificial
  Synthetic primer
acgtagcggatgagaatagagaac

SEQ ID NO: 14 - DNA, 25 bp, Artificial
  Synthetic primer
ctgtgattagatgggtttaccctga

SEQ ID NO: 15 - DNA, 19 bp, Artificial
  Synthetic primer
ccgctccaaggcttcaagg

SEQ ID NO: 16 - DNA, 22 bp, Artificial
  Synthetic primer
atccggttaaagcaggtatggg

SEQ ID NO: 17 - DNA, 24 bp, Artificial
  Synthetic primer
gggatcagagatcggaagaagaaa

SEQ ID NO: 18 - DNA, 23 bp, Artificial
  Synthetic primer
aggaggtctgaacatcatcagtg

SEQ ID NO: 19 - DNA, 21 bp, Artificial
  Synthetic primer
aatcggtgtgttcgcctctac

SEQ ID NO: 20 - DNA, 21 bp, Artificial
  Synthetic primer
cgtagttctcgttccagtcgt

SEQ ID NO: 21 - DNA, 21 bp, Artificial
  Synthetic primer
tacatcggatatctgcggctg

SEQ ID NO: 22 - DNA, 22 bp, Artificial
  Synthetic primer
cggtctgctggggcagtttatc

SEQ ID NO: 23 - DNA, 20 bp, Artificial
  Synthetic primer
ggcacaacaggtagtaggcg

SEQ ID NO: 24 - DNA, 20 bp, Artificial
  Synthetic primer
aagcctcccattcaattgcc

SEQ ID NO: 25 - DNA, 24 bp, Artificial
  Synthetic primer
gagaaaggagacatgtaacaagag

SEQ ID NO: 26 - DNA, 20 bp, Artificial
  Synthetic primer
cctctttgctgctttcacac

SEQ ID NO: 27 - DNA, 19 bp, Artificial
  Synthetic primer
ccgagtctcgtttcctctc

SEQ ID NO: 28 - DNA, 21 bp, Artificial
  Synthetic primer
ctgaaattgcggcagatatac

SEQ ID NO: 29 - DNA, 21 bp, Artificial
  Synthetic primer
ctgatacccaggaagacattc

SEQ ID NO: 30 - DNA, 18 bp, Artificial
  Synthetic primer
gggccaacaccatgttac